(19) United States
(12) Patent Application Publication
(10) Pub. No.: US 2014/0031666 A1
(43) Pub. Date: Jan. 30, 2014

(54) System for Positioning and Holding an Anatomy in a Medical Imaging Device

(71) Applicant: General Electric Company, Schenectady, NY (US)

(72) Inventors: Mohammad Suhail Mohammad Ismail Shaikh, Bangalore (IN); Ravi Shankar Jaiswal, Bangalore (IN); Venkata Prasad Sale Gangi, Bangalore (IN)

(73) Assignee: General Electric Company, Schenectady, NY (US)

(21) Appl. No.: 13/950,092

(22) Filed: Jul. 24, 2013

(30) Foreign Application Priority Data
Jul. 26, 2012 (IN) 3060/CHE/2012

(51) Int. Cl. A61B 6/04

(52) U.S. Cl. CPC A61B 6/04
USPC 600/407

(57) ABSTRACT

A system for positioning an anatomy of a patient in a medical imaging device. The system includes one or more sensor elements disposed on a region of interest of the anatomy movable within a bore of the medical imaging device, and one or more detector units configured to detect the one or more sensor elements for positioning a region of interest of the anatomy with respect to a point in the imaging range of the bore.

### TECHNICAL FIELD

Embodiments of the present invention relate to medical imaging and, more specifically, to a system for positioning and holding an anatomy in a medical imaging device.

### BACKGROUND OF THE INVENTION

Medical imaging techniques are commonly used by hospitals for diagnosing a human body condition. Numerous medical imaging techniques are present and they include for example, radiography, magnetic resonance imaging (MRI), X-ray, fluoroscopy, mammography, ultrasound and positron emission tomography (PET). Further different types of medical imaging devices are also available to cater to various needs. In a medical imaging device such as an MRI device an anatomy that should be scanned needs to be aligned to an imaging range of a bore that receives the anatomy. For example, a medical imaging device may be available to scan an anatomy such as an extremity of a patient. The extremity may be, but not limited to, a leg and a hand. The anatomy is aligned with respect to an iso-center in an imaging range with multiple trial and errors. So in the bore the anatomy position is adjusted by manually placing pads so as to align with respect to the iso-center. However the patient may still move the extremity portion and thus whole process of positioning the anatomy needs to be performed again. Thus, a technician needs to adjust the patient's anatomy such as a fractured hand multiple times causing discomfort to the patient. Moreover this process is cumbersome and time consuming.

Now to focus on a new area of interest on the same anatomy the adjustments again need to be performed manually by the technician. This may render the imaging process to be time consuming. Therefore there is a need for a medical imaging device for capturing images of a patient's anatomy in a convenient manner.

### BRIEF DESCRIPTION OF THE INVENTION

The above-mentioned shortcomings, disadvantages and problems are addressed herein which will be understood by reading and understanding the following specification.

As discussed in detail below, embodiments of the present invention include a system for positioning and holding an anatomy of a patient in a medical imaging device. The system includes one or more sensor elements disposed on a region of interest of the anatomy movable within a bore of the medical imaging device. One or more detector units may be configured to detect the one or more sensor elements for positioning a region of interest of the anatomy with respect to a point in the imaging range within the bore.

In an embodiment of the present invention, a medical imaging device for imaging an anatomy of a patient is disclosed. The medical imaging device includes a bore and one or more detector units. The bore receives the anatomy for imaging. The detector units are configured to detect one or more sensor elements on the anatomy for positioning a region of interest in the anatomy with respect to a point in the imaging range of the bore.

In an embodiment of the present invention, a method of positioning and holding an anatomy of a patient in a medical imaging device is disclosed. The method includes sending signals from one or more detector units to one or more sensor elements disposed on the anatomy. The anatomy is movable into a bore of the medical imaging device. Thereafter the one or more sensor elements are detected for positioning a region of interest in the body extremity with respect to a point in the imaging range of the bore.

### BRIEF DESCRIPTION OF THE DRAWINGS

FIG. 1A and 1B are a schematic illustration of a system for positioning an anatomy of a patient in an medical imaging device 100 in accordance with an embodiment;

FIG. 2 is a schematic illustration of different stages depicting the system that positions the anatomy of the patient in the medical imaging device of FIG. 1;

FIG. 3 is a schematic illustration of a system for positioning the anatomy of the patient in an medical imaging device 100 in accordance with an embodiment;

FIG. 4 is a schematic illustration of a system for positioning an anatomy of a patient in an medical imaging device in accordance with an embodiment;

FIG. 5 is a schematic illustration of a system for positioning an anatomy of a patient in an medical imaging device in accordance with an embodiment;

FIG. 6 is a schematic illustration of the medical imaging device including a holding unit for holding the anatomy within the bore in accordance with an embodiment; and

FIG. 7 is a flowchart of a method of positioning an anatomy of a patient in a medical imaging device in accordance with an embodiment.

### DETAILED DESCRIPTION OF THE INVENTION

In the following detailed description, reference is made to the accompanying drawings that form a part hereof, and in which is shown by way of illustration specific embodiments that may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken as limiting the scope of the invention.

A system for positioning an anatomy of a patient in a medical imaging device is disclosed. The system includes one or more sensor elements disposed on a region of interest of the anatomy movable within a bore of the medical imaging device. One or more detector units may be configured to detect the one or more sensor elements for positioning a region of interest of the anatomy with respect to an iso-center of the bore.

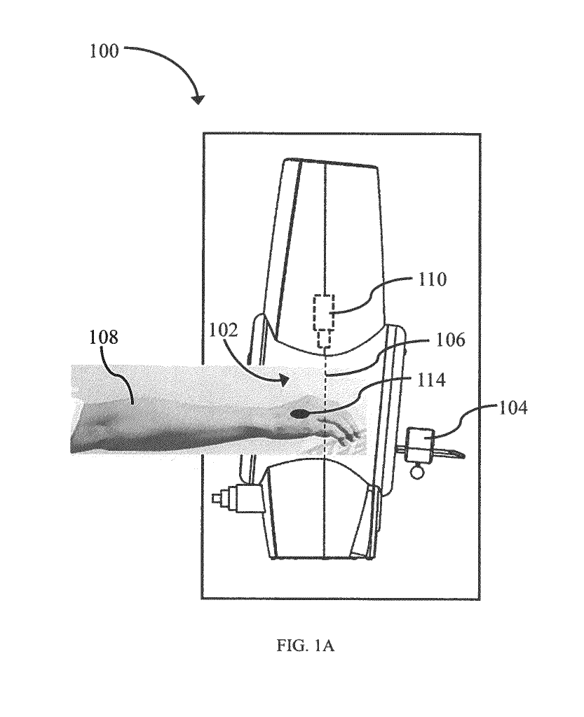

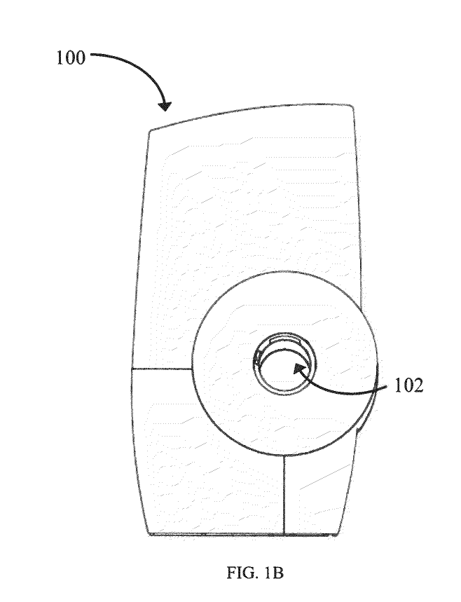

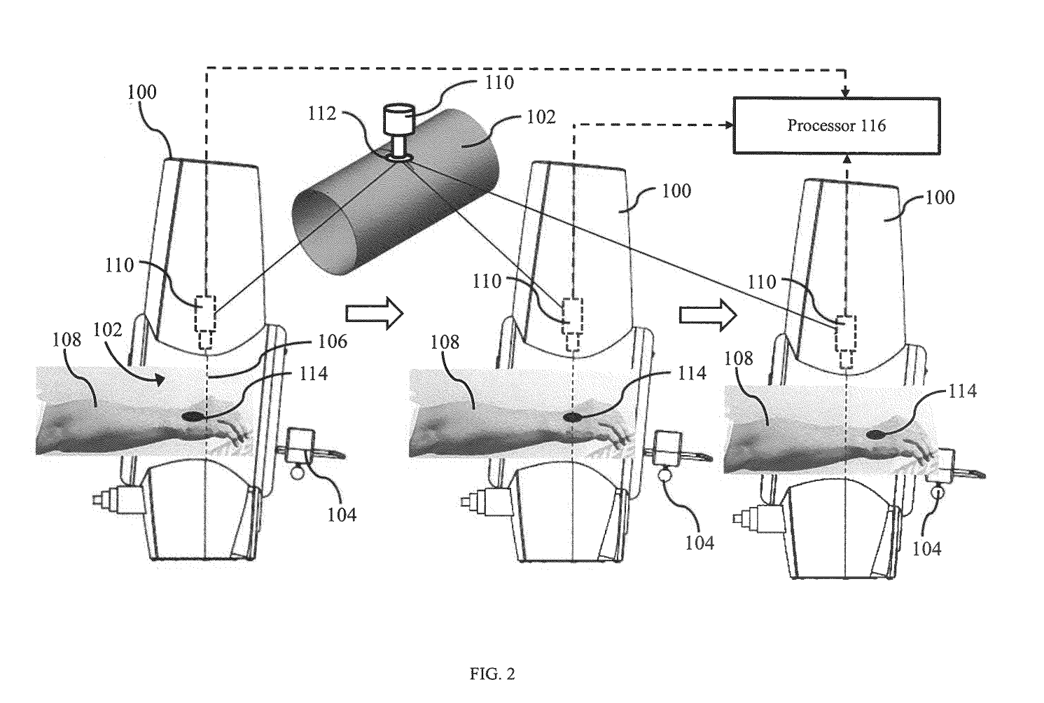

FIG. 1A and FIG. 1B are a schematic illustration of a system for positioning an anatomy of a patient in a medical imaging device 100 in accordance with an embodiment. The medical imaging device 100 may be used to capture an image of an anatomy of a patient. In an embodiment of the present invention, the medical imaging device 100 may be a Magnetic Resonance Imaging (MRI) device. However the medical imaging device 100 may include but not limited to, a radiography device, a MRI device, an X-ray device, a fluoroscopy device, a mammography device, an ultrasound device and Positron Emission Tomography (PET) device. The image may be captured when the anatomy is placed within a bore 102 of the medical imaging device 100. The anatomy may include, but not limited to, a leg and a hand. For example, a patient may place an anatomy, for example a body extremity such as a leg, within the bore 102. The leg may be supported by a supporting unit 104 provided. Further the bore 102 as illustrated in FIG. 2 may have a cylindrical shape. However it may be noted that the bore 102 may have any other shape convenient for receiving the anatomy of the patient. The anatomy can be placed manually or using any movable arrangement present in the medical imaging device 100. Prior to capturing the image, the anatomy needs to be positioned with respect to an imaging range of the bore 102. The imaging range is a region in a bore where the image of an anatomy placed within the bore can be captured. In an embodiment of the present invention, the anatomy may need to be positioned with respect to an iso-center of the bore 102 within the imaging range. For example, in an MRI device an iso-center is the center point of a magnetic field. However it may be envisioned that different types of medical imaging device may have their iso-centers. The iso-center may be present along an axis line 106.

The system illustrated in FIG. 1 facilitates in positioning an anatomy 108, such as a hand, with respect to the imaging range. The system includes one or more detector units such as, a detector unit 110. The detector unit 110 may include, but not limited to, a laser beam detector, and a Light Emitting Diode (LED) detector. In an embodiment of the present invention, the detector unit 110 may include an emitter for emitting a signal on to the anatomy 108 placed within the bore 102 and a detector for detecting the signal reflected from the anatomy 108. The detector unit 110 may be positioned proximal to the bore 102 as illustrated in FIG. 2. The bore 102 may have an opening 112 through which the signal send by the detector unit 110 may travel into the bore 102 and reach the anatomy 108. Further it may be noted that the bore 102 may include more than one opening similar to the opening 112 capable of accommodating other detector units in the medical imaging device 100.

The anatomy 108 may have one or more sensor elements. A sensor element may include one or more sensing markers such as a sensing marker 114 disposed on a region of interest of an anatomy of the anatomy 108. The sensing markers may be similar to a sticker attached to the region of interest. The sensing marker 114 may be for example, but not limited to, an ink marker, a film marker, a strap and a band. The signals send from the detector unit 110 is received at the sensing marker 114 and reflected back to the detector unit 110. The detector unit 110 thus identifies the presence of the sensing marker 114. In order to detect the presence, the sensing marker 114 may need to be aligned along the axis line 106. The axis line 106 may be an imaginary line passing through a center of the detector unit 110.

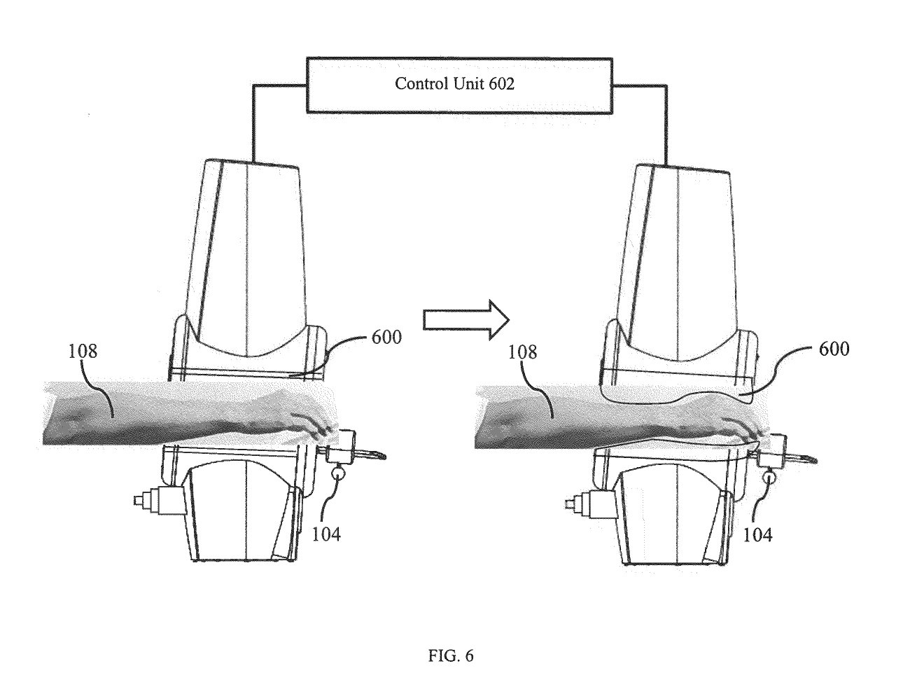

Now during operation for positioning the anatomy 108 with respect to the imaging range of the bore 102, the anatomy 108 may be entered and moved through the bore 102 slowly by the patient as illustrated in FIG. 2. A technician or a medical expert may be present for helping the patient. The detector unit 110 may send signals continuously. In an embodiment of the present invention, the detector unit 110 may send the signals based on the position of the anatomy 108. For example, the detector unit 110 may transmit the signals only when the sensing marker 114 is nearing the imaging range. The signals may fall on the sensing marker 114 positioned on the region of interest such as a palm portion and reflected. The reflected signal is received at the detector unit 110 and it is confirmed that the sensing marker 114 is aligned with the imaging range. In an embodiment of the present invention, the reflected signal may indicate that the sensing marker 114 is aligned to the iso-center within the imaging range. This information is then sent to a processor 116. More specifically, in order to align the sensing marker 114 or the anatomy 108 with respect to the imaging range the adjustments in position need to be performed in multiple axes such as X, Y and Z axes. The Z axis movement refers to a lateral movement or direction of inserting the anatomy 108 through the bore 102. Whereas the movements in X and Y axis refer to the adjustments in position (i.e., in vertical and horizontal directions) of the anatomy 108 made within the bore 102. Now referring back to the reflected signal, the detector unit 110 detects that the sensing marker 114 is aligned with the point in the imaging range in the Z axis. In order to align the sensing marker 114 with respect to the X and Y axes a holding unit may be used. In an embodiment of the present invention, the holding unit may be present within the bore 102. The holding unit is explained in detail in conjunction with FIG. 6.

The processor 116 receives position information of the sensing marker 114 and then informs the patient to stop movement of the anatomy 108 as illustrated in FIG. 2. The patient may be informed using different techniques such, an audio signal, a visual instruction and a combination of both. For instance, the medical imaging device 100 may generate a sound such as, a beep sound audible to the patient so that movement of the anatomy 108 may be stopped. In another instance the medical imaging device 100 may have a display unit capable of presenting visual instructions to the stop the movement of the anatomy 108 when the sensing marker 114 is aligned with the imaging range. The display of visual instructions may be controlled by the processor 116. Further, in an embodiment of the present invention, the processor 116 may also present a visual representation of movement of the anatomy 108 within the bore 102 and alignment of the sensing marker 114 with respect to the imaging range through the display unit. In this case the visual representation may act as instructions to the technician or the patient. However it may be contemplated that the processor 112 may use any other techniques for sending instructions to the patient for stopping the movement of an anatomy in various other embodiments. In case during the movement of the anatomy the sensing marker 114 passes the detector unit 110 then the processor 116 generates an alarm signal. It may be envisioned that the alarm signal may be generated when the sensing marker 114 passes the iso-center by a predefined distance. The predefined distance may vary for different medical imaging devices. Once the alarm signal is generated the processor 116 sends instructions to the patient to move the anatomy back so that the sensing marker 114 can be aligned to the imaging range. In an embodiment the processor 116 may also provide instructions to the patient to move a particular distance backward in order to align the sensing marker 114 with the imaging range.

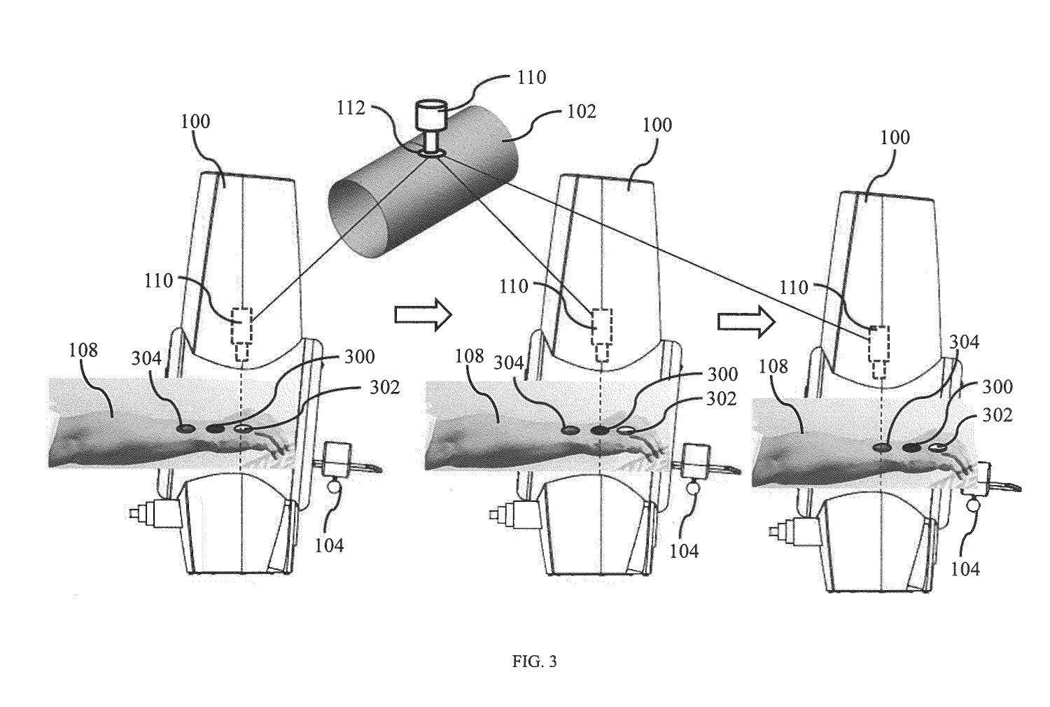

As explained before, there is a possibility of moving the anatomy by the patient within the bore 102 beyond a required limit. So in order to track the movement of the anatomy in a more accurate manner multiple sensing markers may be positioned on the anatomy. FIG. 3 is a schematic illustration of a system for positioning the anatomy of the patient in a medical imaging device 100 in accordance with another embodiment. As illustrated multiple sensing markers such as, a first sensing marker 300, a second sensing marker 302 and a second sensing marker 304 may be placed on the anatomy 108 proximal to each other. For example, on a palm portion of a patient's hand the first sensing marker 300 may be positioned such that it is positioned between the second sensing marker 302 and the second sensing marker 304. However it may be contemplated that the distance between the first sensing marker 300, the second sensing marker 302 and the second sensing marker 304 may be varied in different instances based on a response time requirement of the medical imaging device.

The first sensing marker 300 may be positioned at the region of interest on the anatomy and the second sensing marker 302 and the second sensing marker 304 may be positioned at both sides of the first sensing marker 300. While moving the anatomy 108 within the bore 102 the detector unit 110 may initially detect the second sensing marker 302 when aligned with the point in the imaging range. The processor 116 receives this information to identify that the region of interest is nearing the point in the imaging range. In an embodiment of the present invention, the processor 116 may present this information to the patient and the technician through one of the display units and other techniques such as, a sound signal, an audio and a video. This information indicates that the patient needs to move the anatomy 108 further through the bore 102.

While moving the anatomy 108 the first sensing marker 300 may be detected by the detector unit 110. The processor 116 receives this information and determines that the region of interest of the anatomy 108 is aligned with the imaging range with respect to the Z axis. In an embodiment the processor 116 may detect that the region of interest is aligned with the imaging range only if the first sensing marker 300 may be detectable to the detector unit 110 for a predefined threshold time. For example, a first sensing marker may need to be aligned to a detector unit and detectable for a predefined threshold time. The predefined threshold time may be set by the technician or preset. This may mitigate the possibility of the processor 116 incorrectly registering the presence of region of interest aligned to the imaging range. Such incorrect registration may occur when the first sensing marker 300 is aligned to the imaging range for a small time period and then passes the imaging range due to movement of the anatomy 108. The processor 116 may send instructions to the medical imaging device 100 to align the anatomy 108 in the X and Y axes if required to accurately position the anatomy 108 for imaging. Once positioned the processor 116 instructs an imaging unit (not shown in the FIGS. 1, 2 and 3) to capture an image of the region of interest.

In embodiments of the present invention, the anatomy 108 may move through the bore 102 and the first sensing marker 300 may pass the imaging range. Also the first sensing marker 300 may or may not be detected by the detector unit 110. In this case, in order to realign the first sensing marker 300 to the imaging range the second sensing marker 304 may be utilized. More specifically, when the first sensing marker 300 passes the imaging range the detector unit 110 may detect the second sensing marker 304. The processor 116 receives this information and instructs the patient to move the anatomy 108 so that the first sensing marker 300 is aligned with the imaging range. The processor 116 may provide these instructions in the form of audible signals and/or visual instructions. The additional sensing markers such as the second sensing marker 302 and the second sensing marker 304 facilitate in correcting any errors in detecting the first sensing marker 300 for example, missing of the first sensing marker 300 from detection. Thus, these sensing markers act as check points for determining if the first sensing marker 300 is missed by the detector unit 110.

In an embodiment of the present invention, the sensing markers such as the first sensing marker 300, the second sensing marker 302 and the second sensing marker 304 may have different color codes. For example the first sensing marker 300, the second sensing marker 302 and the second sensing marker 304 may have the color codes green, yellow and/or red. So when the anatomy 108 is moved through the bore 102 the second sensing marker 302 may be detected based on its color code (e.g. yellow). This color code indicates that the region of interest is nearing the imaging range. The processor 116 then sends instructions to the patient to move the anatomy 108 forward. Once the first sensing marker 300 reaches the visibility of the detector unit 110, the first sensing marker 300 is recognized based on the color code (e.g. green). Thereafter the processor 116 notifies the patient to stop moving the anatomy 108. In case the patient moves the anatomy 108 further then the detector unit 110 detects the second sensing marker 304. The color code (e.g. red) of the second sensing marker 304 indicates that the anatomy 108 moved missing the first sensing marker 300. Consequently the processor 116 notifies the patient to move the anatomy 108 such that the first sensing marker 302 is aligned with the detector unit 100. It may be contemplated that different color codes may be attributed to the sensing markers for performing the same functionality in various other embodiments. Moreover, it may be envisioned that any number of sensing markers may be utilized by different other embodiments.

In an embodiment of the present invention, the sensing markers such as the first sensing marker 300, the second sensing marker 302 and the second sensing marker 304 may be capable of storing information indicating a position of the sensing marker on the anatomy 108. For example, as illustrated in FIG. 3, these sensing markers may be placed in a particular order (i.e., the second sensing marker 302, the first sensing marker 300 and the second sensing marker 304 may have first, second and third positions respectively). These sensing markers may indicate their order of placement on the anatomy 108. Thus, while moving the anatomy 108 the detector unit 110 may count the second sensing marker 302 as the first sensing marker. The processor 116 counts the next sensing marker as the first sensing marker 300. When the first sensing marker 300 is detected by the detector unit 110 then the processor 116 determines the region of interest is aligned to the point in the imaging range. Now if the first sensing marker 300 is missed by the detector unit 110 then the second sensing marker 304 may be detected by the detector unit 110 and counted as the third sensing marker. This information enables the processor 116 to identify that the first sensing marker 300 is missed and accordingly notifies the patient to move the anatomy 108 back so that the first sensing marker 300 is detectable.

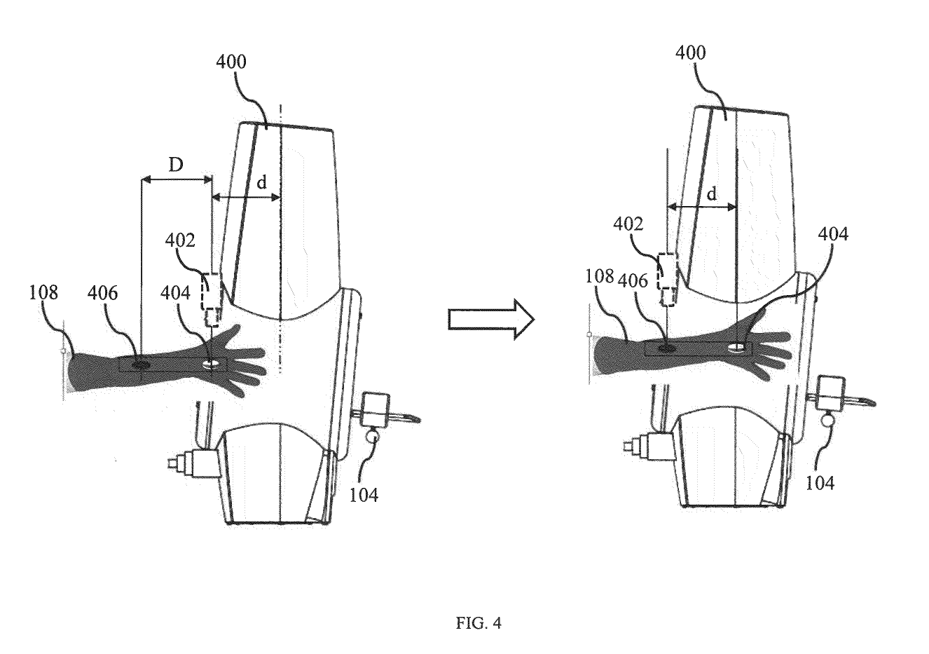

According to an embodiment of the present invention, a system for positioning the anatomy 108 of a patient in a medical imaging device 400 is provided. As illustrated, for example, in FIG. 4, the medical imaging device 400 may include a detector unit 402 placed proximal to an external body of the image device 100. In this embodiment of the present invention, the sensing markers such as a first sensing marker 404 and a second sensing marker 406 may be positioned on the anatomy 108. These sensing markers are placed such that the distance between an imaging range of a bore 408 and the detector unit 402 (e.g. ‘d’) is equal to the distance between the first sensing marker 404 and the second sensing marker 406 (e.g. ‘D’). The distance D is the distance between the first sensing marker 404 and the second sensing marker 406. Whereas the distance ‘d’ is the distance between the detector unit 402 and the point in the image range. The point in the image range may be an iso-center. In an embodiment of the present invention, the first sensing marker 404 and the second sensing marker 406 may have color codes associated with them. During operation when the first sensing marker 404 is detected by the detector unit 402 then the processor 116 receives this information and notifies the patient to move the anatomy 108 by a particular distance. The distance notified is equal to the distance between the point in the imaging range for example an iso-center of the bore 102 and the detector unit 402. The processor 116 may notify the patient in the form of audible and/or visual instructions. The medical imaging device 400 may include a presentation unit such as, but not limited to, a speaker and a display unit for presenting the instructions. The patient may move the anatomy approximately in the prescribed distance so that the second sensing marker 406 is detected by the detector unit 402. The presentation unit may display the movement of the anatomy 108 in the medical imaging device 400. This indicates that the first sensing marker 404 is aligned with respect to the point in the imaging range. Now in case any of the sensing markers are missed from detection, the processor 116 sends notification to the patient for moving the anatomy 108 such that the sensing markers are placed within the visibility of the detector unit 402. Due to the usage of these sensing markers on the anatomy 108 the region of interest may be accurately aligned to the point in the imaging range with minimal trial and error cycles performed manually by the patient or the technician.

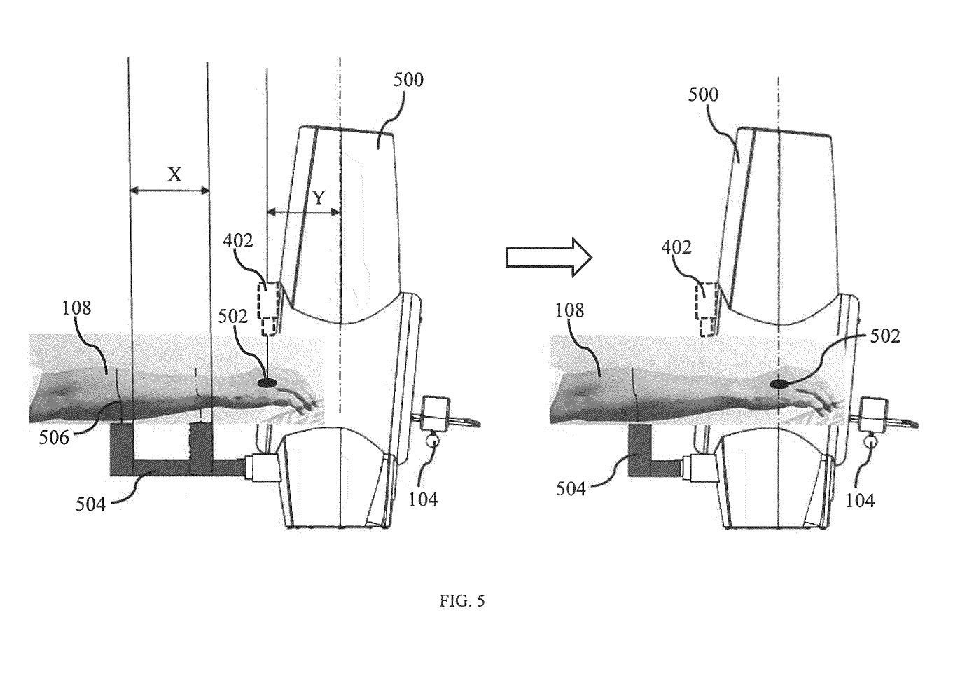

According to an embodiment of the present invention, a system for positioning the anatomy 108 of a patient in the medical imaging device 500 is provided. As illustrated, for example, in FIG. 5, one or more sensing markers such as a sensing marker 502 may be placed on the anatomy 108. The medical imaging device 500 may include a supporting unit 504 for providing a support to the anatomy 108 and also facilitate in moving the anatomy 108 into the bore 102. The supporting unit 504 may be slidably engaged with a body of the medical imaging device 500. The supporting unit 504 may include one or more fastening units such as a fastening unit 506 for holding the anatomy 108. The fastening unit 506 may include, but is not limited to, a belt, a Velcro® unit, and/or an elastic band. Once the patient moves the anatomy 108 the sensing marker 502 may be detected by the detector unit 402. The processor 116 receives this indication and then presents instructions to the patient to move the anatomy 108 further into the bore 102. The patient moves the anatomy 108 into the bore 102 using the supporting unit 504. The supporting unit 504 may move for a distance ‘X’ equal to a distance ‘Y’ between the detector unit 402 and a point in the image range within the bore 102. The distance X refers to a distance between two positions of the supporting unit 504. More specifically, the distance X is a difference in stroke distance between an in-position of the supporting unit 504 and the out-position of the supporting unit 504. Further, the distance Y indicates a distance between the detector unit 402 and the point in the image range. The point in the image range may be an iso-center of the bore 102.

In an embodiment of the present invention, movement of the supporting unit 504 may be restricted to cover the distance between the detector unit 402 and the point in the image range. Thus, once the sensing marker 502 is detected the anatomy 108 may be moved up to a distance to align the sensing marker 502 with the point in the image range using the supporting unit 504. In an embodiment of the present invention, the supporting unit 504 may automatically slide once the sensing marker 502 is detected by the detector unit 402 to align the sensing marker 502 with the point in the image range. In this scenario, the processor 116 may detect that the sensing marker 502 is aligned with the detector unit 402 and instruct the supporting unit 504 to move. The movement of the supporting unit 504 may be preset. In an embodiment of the present invention, the medical imaging device 500 may include a presentation unit (not shown in FIG. 5) for enabling a technician to set a distance for moving the supporting unit 504. Once the distance is set, the supporting unit 504 moves to align the sensing marker 502 with respect to the point in the image range. Moreover, the supporting unit 504 may be capable of automatically moving the anatomy 108 in any other direction for aligning the sensing marker 502 with the point in the image range.

With reference to FIG. 1, in order to align the anatomy 108 with respect to the iso-center, the position of the anatomy 108 may need to be adjusted in the X and Y axes. FIG. 6 is a schematic illustration of the medical imaging device 100 including a holding unit 600 for holding the anatomy 108 within the bore 102. When the anatomy 108 placed within the bore 102 is aligned to the point in the imaging range with respect to the Z axis, in order to align the anatomy 108 with respect to the X and Y axes the holding unit 600 is used. In an embodiment of the present invention, the holding unit 600 may be a bladder that is expandable. The bladder may be for example a tube and balloon. Thus, the bladder may be expanded in response to instructions received from the processor 116. The processor 116 may send an instruction to a control unit 602 when the region of interest or the sensing marker 114 (not shown in FIG. 6) is aligned to the point in the imaging range. The control unit 602 may include but not limited to an electrical circuitry and processor for processing the instructions received from the processor 116. In an instance, the expansion of the bladder may be initiated upon receiving a user input. The user input may be received through a presentation unit present in the medical imaging device 100. The presentation unit may have a touch interface.

In an embodiment of the present invention, air may be supplied into the bladder for expansion. Once expanded, the bladder positions the anatomy 108 aligned to the point in the imaging range accurately also in the X and Y axes. The expanded bladder also holds the anatomy 108 tightly in the position so that there is no change in the position due movement of the anatomy 108. In an embodiment of the present invention, an amount of air supplied to the bladder for expansion (i.e. air pressure) may be controlled based on instructions from the processor 116. The amount of air needed is dependent on the adjustments required in the X and Y axes for accurately positioning the bladder in the bore 102. In an embodiment of the present invention, the bladder may be expanded using any other sources. In various other embodiments of the present invention, it may be contemplated that different types of holding units may be used for holding the patient within the bore 102.

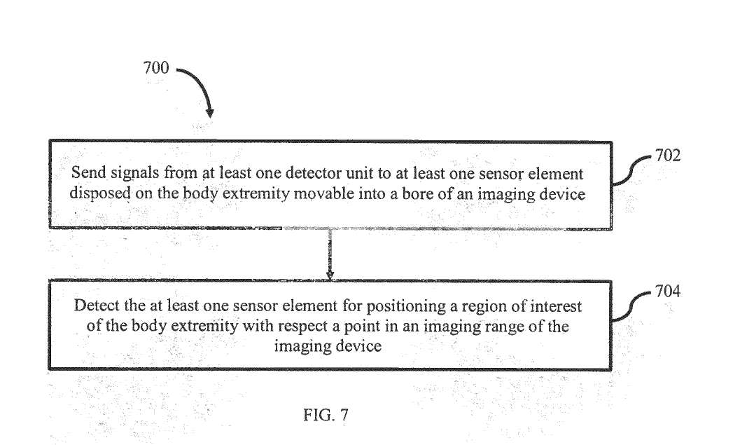

According to an embodiment of the present invention, a method of positioning an anatomy of a patient in a medical imaging device is provided. As illustrated, for example, in FIG. 7, the method involves sending signals from one or more detector units to one or more sensor elements disposed on the anatomy of the patient at step 702. The detector unit may be placed proximal to a bore. In this case the bore may have an opening for allowing the signals from the detector unit to pass into the bore. This is explained in detail in conjunction, for example, with FIG. 2 and FIG. 3. The anatomy may be movable within the bore of the medical imaging device. The signals passing through the opening may be received by a sensor element placed on the anatomy. The sensor element may include multiple sensing markers. When the signals are received at a sensing marker they are reflected back and received by the detector unit. This facilitates in identifying the presence of the sensing marker. In an embodiment of the present invention, the presence of the sensing marker is detected only when the sensing marker is detectable to the detector unit for a predefined threshold time. In a scenario, the detector unit may transmit the signals only when the sensing marker is nearing the imaging range. The signals may fall on the sensing marker positioned on the region of interest such as a palm portion and reflected. The reflected signal is received at the detector unit and it is confirmed that the sensing marker is aligned with the imaging range. In an embodiment of the present invention, the reflected signal may indicate that the sensing marker is aligned to the point in the imaging range.

The presence of the sensor marker facilitates in positioning the region of interest with respect to the point in the imaging range of the bore at step 704. In an embodiment of the present invention, in case the identified sensing marker is placed on a region of interest on the anatomy, then this indicates that the region of interest is aligned with the point in the imaging range. In an embodiment of the present invention, if the detector unit positioned on a body of the medical imaging device and detects the presence of the sensing marker then the anatomy is moved to align with the point in the imaging range. A distance of movement is equal to a distance between the detector unit and the point in the imaging range.

Moreover in order to align the sensing marker or the anatomy with respect to the imaging range the adjustments in position need to be performed in multiple axes such as X, Y and Z axes. The Z axis movement refers to a lateral movement or direction of inserting the anatomy through the bore. Whereas the movements in X and Y axes refer to the adjustments in position (i.e., in vertical and horizontal directions) of the anatomy made within the bore. Now referring back to the reflected signal, the detector unit detects that the sensing marker is aligned with the point in the imaging range in the Z axis. In order to align the sensing marker with respect to the X and Y axes, a holding unit may be used.

The method 700 can be performed using a processor or any other processing device. The method steps can be implemented using coded instructions (e.g., computer readable instructions) stored on a tangible computer readable medium. The tangible computer readable medium may be for example a flash memory, a read-only memory (ROM), a random access memory (RAM), any other computer readable storage medium and any storage media. Although the method of positioning an anatomy of a patient in a medical imaging device is explained with reference to the flow chart of FIG. 7, other methods of implementing the method can be employed. For example, the order of execution of each method steps may be changed, and/or some of the method steps described may be changed, eliminated, divide or combined. Further the method steps may be sequentially or simultaneously executed for displaying a medical image film on a display unit of a patient monitoring device.

This written description uses examples to disclose the invention, including the best mode, and also to enable any person skilled in the art to practice the invention, including making and using any computing system or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A system for positioning an anatomy of a patient in a medical imaging device, the system comprising:
at least one sensor element disposed on the anatomy, wherein the anatomy is movable within a bore of the medical imaging device; and
at least one detector unit configured to detect the at least one sensor element to position a region of interest of the anatomy with respect to an imaging range of the bore of the medical imaging device.

2. The system of claim 1, wherein a sensor element of the at least one sensor element comprises a plurality of sensing markers.

3. The system of claim 2, wherein a detector unit of the at least one detector unit detects a sensing marker in response to identifying the presence of the sensing marker for a predefined threshold time.

4. The system of claim 1, wherein a sensor element of the at least one sensor element comprises:
a first sensing marker disposed at the region of interest; and
at least one second sensing marker positioned proximal to the first sensing marker, wherein the at least one second sensing marker assists in positioning the first sensing marker with respect to the imaging range of the bore.

5. The system of claim 1, wherein a detector unit of the at least one detector unit is positioned within the imaging range of the bore.

6. The system of claim 1, wherein a detector unit of the at least one detector unit is positioned proximal to an end of the bore.

7. The system of claim 6, wherein a sensor element of the at least one sensor element comprises:
a first sensing marker disposed at the region of interest; and
a second sensing marker disposed proximal to the first sensing marker positioned at the region of interest, wherein a distance between the first sensing marker and the second sensing marker is equal to a distance between the detector unit and a point in the imaging range.

8. The system of claim 7, further comprising a holding unit configured to hold the anatomy within the bore in response to a detection of the region of interest as aligned to an iso-center of the bore, wherein the iso-center is the point in the imaging range.

9. The system of claim 8, further comprising a presentation unit configured to indicate to a user the region of interest as aligned with respect to the iso-center.

10. A medical imaging device for imaging an anatomy of a patient, the medical imaging device comprising:
a bore configured to receive the anatomy for imaging; and
at least one detector unit configured to detect at least one sensor element disposed on the anatomy to position a region of interest of the anatomy with respect to an imaging range of the bore.

11. The medical imaging device of claim 10, wherein at least one sensor element of the at least one sensor element comprises:
a first sensing marker disposed at the region of interest; and
a second sensing marker disposed proximal to the first sensing marker, wherein a distance between the first sensing marker and the second sensing marker is equal to a distance between the detector unit and a point in the imaging range.

12. The medical imaging device of claim 10, wherein a detector unit of the at least one detector unit is positioned within the imaging range of the bore.

13. The medical imaging device of claim 12, wherein the bore comprises an opening to enable communication of signals between the at least one detector unit and the at least one sensor element.

14. The medical imaging device of claim 10, wherein a detector unit of the at least one detector unit is positioned proximal to an end of the bore.

15. The medical imaging device of claim 14, wherein at least one sensor element of the at least one sensor element comprises:
a first sensing marker positioned at the region of interest of the anatomy; and
a second sensing marker positioned proximal to the first sensing marker, wherein a distance between the first sensing marker and the second sensing marker is equal to a distance between a detector unit of the at least one detector unit and a point in the imaging range.

16. The medical imaging device of claim 14, further comprising a supporting unit configured to support the anatomy and to move in order to enable alignment of the region of interest with respect to an iso-center, where the iso-center is a point in the imaging range, wherein subsequent to a detection of the sensor element by a detector unit of the at least one detector unit, a distance of movement of the supporting unit is equal to a distance between the iso-center and the detector unit.

17. The medical imaging device of claim 10, further comprising a holding unit configured to hold the anatomy within the bore in response to a detection that the region of interest is positioned within the imaging range.

18. The medical imaging device of claim 10, further comprising a presentation unit configured to indicate to a user when the region of interest is positioned within the imaging range.

19. A method of positioning an anatomy of a patient in an medical imaging device, the method comprising:
sending signals from at least one detector unit to at least one sensor element disposed on the anatomy movable into a bore of the medical imaging device; and
detecting the at least one sensor element for positioning a region of interest of the anatomy within an imaging range of the bore.

20. The method of claim 19, further comprising:
identifying a sensor element of the at least one sensor element present in the region of interest as aligned to an iso-center for a predefined threshold time, wherein the iso-center is within the imaging range of the bore; and
determining the region of interest is aligned to the iso-center.
